SEQUENCE LISTING (4 sequences)

SEQ ID NO: 1 - protein, 738 aa, Adeno Associated Virus
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAAPSGVGPNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 2 - protein, 736 aa, Adeno Associated Virus
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL

SEQ ID NO: 3 - DNA, 1672 bp, Artificial Sequence
  CAG Promoter Sequence
atagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaa

SEQ ID NO: 4 - protein, 563 aa, Homo sapiens
MGLQACLLGLFALILSGKCSYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVVRSPHPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNP

(19) United States
(12) Patent Application Publication
(10) Pub. No.: US 2022/0184188 A1
(43) Pub. Date: Jun. 16, 2022

(54) AAV VECTOR TREATMENT METHODS FOR LATE INFANTILE NEURONAL CEROID LIPOFUSCINOSIS TYPE 2

(71) Applicant: SPARK THERAPEUTICS, INC., Philadelphia, PA (US)

(72) Inventors: David William ANDERSON, Newtown Square, PA (US); Maryann TOTO, Wilmington, DE (US); Sue E.I. DASEN, Ardmore, PA (US)

(73) Assignee: SPARK THERAPEUTICS, INC., Philadelphia, PA (US)

(21) Appl. No.: 17/310,378

(22) PCT Filed: Jan. 31, 2020
(86) PCT No.: PCT/US2020/016235

§ 371 Date: Jul. 29, 2021

Related U.S. Application Data
(60) Provisional application No. 62/800,131, filed on Feb. 1, 2019.

(51) Int. Cl. A61K 38/48; A61K 48/00; A61P 37/06; C12N 15/86

(52) U.S. Cl. CPC A61K 38/4813; A61K 48/005; A61K 48/0075; C12N 2750/14143; C12N 15/86; C12Y 304/14009; A61P 37/06

(57) ABSTRACT

Disclosed herein are methods for treating a primate in need of tripeptidyl peptidase 1 (TPP1), comprising (a) providing a recombinant adeno-associated virus (AAV) vector comprising a nucleic acid encoding TPP1; and (b) administering an amount of the recombinant AAV vector to the central nervous system (CNS) of the primate, wherein the TPP1 is expressed in the primate.

### RELATED APPLICATIONS

This patent application claims the benefit of priority to U.S. Provisional Patent Application No. 62/800,131, filed on Feb. 1, 2019. The entire content of the foregoing applications is incorporated herein by reference, including all text, tables, drawings and sequences.

### INTRODUCTION

Late infantile Neuronal Ceroid Lipofuscinosis type 2 (CLN2), also referred to as Jansky-Bielschowsky disease and late infantile NCL (LINCL), is a progressive neurodegenerative disease that presents in children around the age of about 2 to 4 years. Symptoms include seizures, loss of motor control and vision, cognitive and developmental impairment, culminating in death within the first two decades of life. The underlying pathological mechanism is a deficiency or defect of the soluble lysosomal enzyme tripeptidyl peptidase-1 (TPP1), due to mutations in the corresponding gene.

Reports indicate that adeno-associated virus (AAV) vector transduction of ependymal cells lining the lateral ventricles of the brain can provide continuous secretion of human TPP1 into the cerebrospinal fluid (CSF), thereby delivering the expressed TPP1 protein across the entire central nervous system (Martz, L., Biocentury Innovation, Dec. 10, 2015). Delivery of AAV2-CAG-TPP1 via ependymal transduction in a canine model of CLN2 was reported to provide disease modification and extension of life (Katz, M. L., et al. (2015). Sci Transl Med, 7(313)).

### SUMMARY

Disclosed herein are non-human primate studies assessing safety and tolerability of an AAV2-CAG-humanTPP1 vector. The AAV vector was delivered by unilateral injection into the lateral ventricle at 3 doses ranging from 1E13 to 2.17E14 vector genomes/brain, followed by 5 and 20 weeks observation. Changes in TPP1 activity and antigen levels in CSF from baseline in each animal were monitored. TPP1 activity levels showed peak increases compared to baseline from ˜17-fold in the low dose cohort, to ˜48-fold in the high dose cohort. Furthermore, average hTPP1 transgene expression levels at all doses tested exceeded Kᵤₚₜₐₖₑ ranges for TPP1 through the duration of the study. Preliminary analysis of relevant central nervous system (CNS) tissues has identified no pathological changes associated with the delivery of the vector or expression of the TPP1 transgene. In conclusion, expression of human TPP1 following ependymal transduction utilizing an AAV2 vector in non-human primates was well tolerated, provided sustained CSF TPP1 protein expression consistently within or exceeding the Kᵤₚₜₐₖₑ value of about 60 to about 120 ng/mL sufficient to provide a therapeutic effect to animals with CLN2.

In certain embodiments, a method of treating a primate in need of tripeptidyl peptidase 1 (TPP1), comprising (a) providing a recombinant adeno-associated virus (AAV) vector comprising a nucleic acid encoding TPP1; and (b) administering an amount of the recombinant AAV vector to the central nervous system (CNS) of the primate, wherein the TPP1 is expressed in the primate.

In certain embodiments, the primate is a human. In certain embodiments, the human has late infantile neuronal ceroid lipofuscinosis (CLN2). In certain embodiments, the human is approximately 1-10 years old or is older than 10 years. In certain embodiments, the human is approximately 2-5 years old.

In certain embodiments, in methods of treating a primate, the recombinant AAV vector is administered to lateral ventricle or cisternae magna. In certain embodiments, the recombinant AAV vector is administered to occipital horn of the lateral ventricle. In certain embodiments, the recombinant AAV vector is unilaterally administered to one lateral ventricle. In certain embodiments, the recombinant AAV vector is bilaterally administered to each lateral ventricle. In certain embodiments, the recombinant AAV vector is unilaterally or bilaterally administered to one or both lateral ventricles multiple times.

In certain embodiments, the TPP1 is expressed at increased levels in the CNS. In certain embodiments, the TPP1 is expressed or delivered throughout the CNS. In certain embodiments, the TPP1 is expressed in or delivered to ependymal cells. In certain embodiments, the TPP1 is delivered to parenchyma.

In certain embodiments, TPP1 expression is sustained at levels equal to or greater than required for half maximal TPP1 uptake into neurons. In certain embodiments, TPP1 expression is sustained at levels equal to or greater than Kᵤₚₜₐₖₑ, wherein Kᵤₚₜₐₖₑ is at least about 60 ng/mL. In certain embodiments, TPP1 expression is sustained at levels equal to or greater than Kᵤₚₜₐₖₑ, wherein Kᵤₚₜₐₖₑ is at least about 60 ng/mL-120 ng/mL. In certain embodiments, TPP1 expression is sustained at levels greater than about 120 ng/mL. In certain embodiments, TPP1 expression is sustained at levels greater than about 150 ng/mL, greater than about 200 ng/mL, greater than about 250 ng/mL or greater than about 300 ng/mL. In certain embodiments, TPP1 expression is sustained for at least about 5 weeks, or at least about 10 weeks, or at least about 20 weeks in the CNS. In certain embodiments, detectable TPP1 expression or TPP1 activity is sustained for at least 5 weeks, or at least 10 weeks, or at least 20 weeks in the CNS.

In certain embodiments, in methods of treating a primate, the recombinant AAV vector is administered to the CNS at a dose of greater than about 1.5×10¹³ AAV vector genomes; at a dose of about 5×10¹³ AAV vector genomes or greater than about 5×10¹³ AAV vector genomes; at a dose of about 1×10¹⁴ AAV vector genomes or greater than about 1×10¹⁴ AAV vector genomes; at a dose of about 5×10¹⁴ AAV vector genomes or greater than about 5×10¹⁴ AAV vector genomes; at a dose of about 1×10¹⁵ AAV vector genomes or greater than about 1×10¹⁵ AAV vector genomes; or at a dose of about 5×10¹⁵ AAV vector genomes or greater than about 5×10¹⁵ AAV vector genomes.

In certain embodiments, in methods of treating a primate, the recombinant AAV vector is administered to the CNS at a dose range from about 1.5×10¹³ to about 5×10¹⁵ vector genomes; at a dose range from about 1×10¹⁴ to about 3×10¹⁵ vector genomes; at a dose range from about 2×10¹⁴ to about 2×10¹⁵ vector genomes; at a dose range from about 2.5×10¹⁴ to about 7.5×10¹⁴ vector genomes; at a dose range from about 5×10¹⁴ to about 5×10¹⁵ vector genomes; or at a dose range from about 1×10¹⁵ to about 5×10¹⁵ vector genomes.

In certain embodiments, in methods of treating a primate, the recombinant AAV vector is administered to the CNS at a dose of about 1×10¹⁴ vector genomes, at a dose of about 2×10¹⁴ vector genomes, at a dose of about 3×10¹⁴ vector genomes, at a dose of about 4×10¹⁴ vector genomes, at a dose of about 5×10¹⁴ vector genomes, at a dose of about 6×10¹⁴ vector genomes, at a dose of about 7×10¹⁴ vector genomes, at a dose of about 8×10¹⁴ vector genomes, at a dose of about 9×10¹⁴ vector genomes, at a dose of about 1×10¹⁵ vector genomes, at a dose of about 2×10¹⁵ vector genomes, at a dose of about 3×10¹⁵ vector genomes, at a dose of about 4×10¹⁵ vector genomes, or at a dose of about 5×10¹⁵ vector genomes.

In certain embodiments, the method reduces, decreases or inhibits one or more symptoms of CLN2; or prevents or reduces progression or worsening of one or more symptoms of CLN2; or stabilizes one or more symptoms of CLN2; or improves one or more symptoms of CLN2.

In certain embodiments, the one or more symptoms is selected from the group consisting of vision impairment, impaired or stunted cognitive development, loss of motor control and seizures.

In certain embodiments, the nucleic acid encoding TPP1 comprises an expression cassette operably linked to an expression control element. In certain embodiments, the expression control element is positioned 5′ of the nucleic acid. In certain embodiments, the expression control element comprises a CAG (SEQ ID NO:3) promoter, cytomegalovirus (CMV) immediate early promoter/enhancer, Rous sarcoma virus (RSV) promoter/enhancer, SV40 promoter, dihydrofolate reductase (DHFR) promoter, or chicken β-actin (CBA) promoter.

In certain embodiments, the heterologous nucleic acid is positioned between one or more 5′ and/or 3′ AAV inverted terminal repeats (ITR(s)). In certain embodiments, the one or more 5′ and/or 3′ AAV ITR(s) comprises a mutated, modified or variant AAV ITR that is not processed by AAV Rep protein. In certain embodiments, the one or more 5′ and/or 3′ AAV ITR(s) comprises a mutated, modified or variant AAV ITR that allows or facilitates formation of the self-complementary reporter transgene genome into a double strand inverted repeat sequence structure in the recombinant AAV vector. In certain embodiments, the mutated, modified or variant AAV ITR has a deleted D sequence, and/or a mutated, modified or variant terminal resolution site (TRS) sequence.

In certain embodiments, the recombinant AAV vector comprises in 5′ 4 3′ orientation a first AAV ITR; a promoter operable in mammalian cells; the heterologous nucleic acid; a polyadenylation signal; and optionally a second AAV ITR.

In certain embodiments, the one or more ITR(s) comprises AAV serotype AAV1, AAV2, AAV3, AAV3B, AAV4, AAV5, AAV6, AAV7, AAV8, AAV9, AAV10, AAV11, AAV12, Rh74 or Rh10 ITR.

In certain embodiments, the recombinant AAV vector comprises a VP1, VP2 or VP3 sequence 60% or more identical to a VP1, VP2 and/or VP3 sequence of AAV serotype AAV1, AAV2, AAV3, AAV3B, AAV4, AAV5, AAV6, AAV7, AAV8, AAV9, AAV10, AAV11, AAV12, Rh74, Rh10, SPK1 (SEQ ID NO:1), or SPK2 (SEQ ID NO:2) VP1, VP2 and/or VP3, or a hybrid or chimera of any of the foregoing AAV serotypes. In certain embodiments, the recombinant AAV vector comprises VP1, VP2 and/or VP3 capsid protein having 100% sequence identity to VP1, VP2 and/or VP3 capsid protein selected from the group consisting of AAV1, AAV2, AAV3, AAV3B, AAV4, AAV5, AAV6, AAV7, AAV8, AAV9, AAV10, Rh10, Rh74, SPK1 (SEQ ID NO:1) and SPK2 (SEQ ID NO:2) VP1, VP2 and/or VP3 capsid proteins.

In certain embodiments, the recombinant AAV vector further comprises a polyadenylation sequence positioned 3′ of the nucleic acid. In certain embodiments, the nucleic acid encoding TPP1, expression control element or polyadenylation sequence is CpG reduced compared to wild-type nucleic acid encoding TPP1, expression control element or polyadenylation sequence. In certain embodiments, the polyadenylation sequence comprises a bovine growth hormone (bGH) polyadenylation sequence.

In certain embodiments, the TPP1 is human, comprises or consists of the sequence set forth as SEQ ID NO:4, or is a functional variant or polymorphic form thereof.

In certain embodiments, the recombinant AAV vector comprises (a) one or more of an AAV capsid, and (b) one or more AAV inverted terminal repeats (ITR(s)), wherein the one or more AAV ITR(s) flanks the 5′ or 3′ terminus of the nucleic acid or the expression cassette.

In certain embodiments, the recombinant AAV vector further comprises an intron positioned 5′ or 3′ of the one or more ITR(s).

In certain embodiments, at least one or more of the one or more ITR(s) and/or the intron is modified to have reduced CpGs.

In certain embodiments, the recombinant AAV vector has a capsid serotype comprising an AAV VP1, VP2 and/or VP3 capsid having 90% or more sequence identity to AAV1, AAV2, AAV3, AAV3B, AAV4, AAV5, AAV6, AAV7, AAV8, AAV9, AAV10, AAV11, Rh10, Rh74, AAV-2i8, SPK1 (SEQ ID NO:1), or SPK2 (SEQ ID NO:2) VP1, VP2 and/or VP3 sequences, or a capsid having 95% or more sequence identity to AAV1, AAV2, AAV3, AAV3B, AAV4, AAV5, AAV6, AAV7, AAV8, AAV9, AAV10, AAV11, AAV12, Rh10, Rh74, AAV-2i8, SPK1 (SEQ ID NO:1), SPK2 (SEQ ID NO:2) VP1, VP2 and/or VP3 sequences, or a capsid having 100% sequence identity to AAV1, AAV2, AAV3, AAV3B, AAV4, AAV5, AAV6, AAV7, AAV8, AAV9, AAV10, AAV11, Rh10, Rh74, AAV-2i8, SPK1 (SEQ ID NO:1), or SPK2 (SEQ ID NO:2) VP1, VP2 and/or VP3 sequences.

In certain embodiments, the one or more ITR(s) comprises one or more ITRs of any of: AAV1, AAV2, AAV3, AAV3B, AAV4, AAV5, AAV6, AAV7, AAV8, AAV9, AAV10, AAV11, Rh10, or Rh74 AAV serotypes, or a combination thereof.

In certain embodiments, the recombinant AAV vector is in a pharmaceutical composition comprising a biologically compatible carrier or excipient.

In certain embodiments, the pharmaceutical composition further comprises empty AAV capsids. In certain embodiments, the ratio of the empty AAV capsids to the recombinant AAV vector is within or between about 100:1-50:1, from about 50:1-25:1, from about 25:1-10:1, from about 10:1-1:1, from about 1:1-1:10, from about 1:10-1:25, from about 1:25-1:50, or from about 1:50-1:100. In certain embodiments, the ratio of the empty AAV capsids to the recombinant AAV vector is about 2:1, 3:1, 4:1, 5:1, 6:1, 7:1, 8:1, 9:1, or 10:1.

In certain embodiments, the pharmaceutical composition further comprises a surfactant.

### DESCRIPTION OF DRAWINGS

FIG. 1 shows a representative magnetic resonance imaging (MRI) image of the targeting of the occipital horn of lateral ventricle (white vertical line).

FIGS. 2A and 2B show rapid expression of human TPP1 protein in the CSF. (A) Human TPP1 levels in the CSF for 30 days following vector administration. All doses (1.0×10¹³ vg, 5.0×10¹³ vg and 2.17×10¹⁴ vg per animal) provided measurable increases in TPP1 protein levels from 2 weeks post transduction. Asterix (*) indicates samples that were hemolyzed, potentially elevating outcomes. (B) Analysis of human TPP1 activity levels in CSF indicated functional protein expression.

FIGS. 3A and 3B show sustained human TPP1 protein expression and activity in the CSF over 20 weeks. (A) Human TPP1 levels in the CSF over 20 weeks, following AAV2-CAG-hTPP1 delivery. Levels of hTPP1 expression exceeded the level required for half maximal uptake into neuron lysosomes (Kᵤₚₜₐₖₑ is approximately 60-120 ng/mL) in all but one animal Expression levels for the 5.0×10¹³ vg/animal dose averaged 1.55-fold above the upper bounds of Kᵤₚₜₐₖₑ at the end of the study and showed relatively consistent expression from week 10-20 on a per animal basis. (B) Human TPP1 activity levels in CSF. All animals that showed sustained expression of human TPP1 in the CSF maintained elevated levels of TPP1 activity throughout the duration of the time-course. As was seen for TPP1 protein expression, the average activity level was found to be highest in the animals that received a dose of 5.0×10¹³ vg/animal.

FIG. 4 shows average levels of TPP protein expression in CSF throughout the duration of the time-course.

### DETAILED DESCRIPTION

The TPP1 “polypeptides,” “proteins” and “peptides” encoded by a “nucleic acid” or “polynucleotide” sequences,” include full-length native TPP1 sequences, as with naturally occurring wild-type TPP1 proteins, as well as functional TPP1 subsequences, modified forms or sequence variants so long as the subsequence, modified form or variant retain some degree of functionality of the native full-length TPP1 protein. In methods and uses of the invention, such TPP1 polypeptides, proteins and peptides encoded by the nucleic acid sequences can be but are not required to be identical to the endogenous TPP1 protein that is defective, or whose expression is insufficient, or deficient in the treated mammal.

A TPP1 polypeptide or TPP1 encoding polynucleotide can include one or more amino acid residue or nucleotide modification, respectively, for example and without limitation, one or more amino acid residue or nucleotide substitution (e.g., 1-3, 3-5, 5-10, 10-15, 15-20, 20-25, 25-30, 30-40, 40-50, 50-100, 100-150, 150-200, 200-250, 250-500, 500-750, 750-850 or more amino acid residues or nucleotides).

An example of an amino acid modification is a conservative amino acid substitution or a deletion (e.g., subsequences or fragments) of a reference sequence, e.g. in TPP1. In certain embodiments, a modified or variant TPP1 sequence retains at least part of a function or activity of unmodified TPP1 sequence.

All mammalian and non-mammalian forms of nucleic acids encoding TPP1, including other mammalian forms of the TPP1 are expressly included, either known or unknown.

As used herein, the term “vector” refers to small carrier nucleic acid molecule, a plasmid, virus (e.g., AAV vector), or other vehicle that can be manipulated by insertion or incorporation of a nucleic acid. Such vectors can be used for genetic manipulation (i.e., “cloning vectors”), to introduce/transfer polynucleotides into cells, and to transcribe or translate the inserted polynucleotide in cells. An “expression vector” is a specialized vector that contains a gene or nucleic acid sequence with the necessary regulatory regions needed for expression in a host cell.

A vector nucleic acid sequence generally contains at least an origin of replication for propagation in a cell and optionally additional elements, such as a heterologous nucleic acid (e.g., nucleic acid encoding TPP1), expression control element (e.g., a promoter, enhancer), intron, an inverted terminal repeat (ITR), selectable marker (e.g., antibiotic resistance), polyadenylation signal.

A viral vector is derived from or based upon one or more nucleic acid elements that comprise a viral genome. Particular viral vectors include adeno-associated virus (AAV) and lentiviral vectors.

The term “recombinant,” as a modifier of vector, such as recombinant AAV (rAAV) vector, as well as a modifier of sequences such as recombinant nucleic acids and polypeptides, means that the compositions have been manipulated (i.e., engineered) in a fashion that generally does not occur in nature. A particular example of a recombinant AAV vector would be where a nucleic acid sequence that is not normally present in the wild-type AAV genome is inserted within the AAV genome. Although the term “recombinant” is not always used herein in reference to AAV vectors, as well as sequences such as nucleic acids, recombinant forms including polynucleotides, are expressly included in spite of any such omission.

A “recombinant AAV vector” or “rAAV” is derived from the wild type genome of AAV by using molecular methods to remove the wild type genome from the AAV genome, and replacing with a non-native nucleic acid sequence, referred to as a heterologous nucleic acid. Typically, for AAV one or both inverted terminal repeat (ITR) sequences of AAV genome are retained in the AAV vector. rAAV is distinguished from an AAV genome, since all or a part of the AAV genome has been replaced with a non-native (non-AAV) sequence with respect to the AAV genomic nucleic acid. Incorporation of a non-native sequence therefore defines the AAV vector as a “recombinant” vector, which can be referred to as a “rAAV vector.”

A rAAV sequence can be packaged—referred to herein as a “particle”—for subsequent infection (transduction) of a cell, ex vivo, in vitro or in vivo. Where a recombinant AAV vector sequence is encapsidated or packaged into an AAV particle, the particle can also be referred to as a “rAAV vector” or “rAAV particle.” Such rAAV particles include proteins that encapsidate or package the vector genome and in the case of AAV, they are referred to as capsid proteins.

A “vector genome” or conveniently abbreviated as “vg” refers to the portion of the recombinant plasmid sequence that is ultimately packaged or encapsidated to form a viral (e.g., rAAV) particle. In cases where recombinant plasmids are used to construct or manufacture recombinant vectors, the vector genome does not include the portion of the “plasmid” that does not correspond to the vector genome sequence of the recombinant plasmid. This non vector genome portion of the recombinant plasmid can be referred to as the “plasmid backbone,” which is important for cloning and amplification of the plasmid, a process that is needed for propagation and recombinant virus production, but is not itself packaged or encapsidated into virus (e.g., AAV) particles. Thus, a “vector genome” refers to the nucleic acid that is packaged or encapsidated by virus (e.g., AAV).

As used herein, the term “serotype” in reference to an AAV vector means a capsid that is serologically distinct from other AAV serotypes. Serologic distinctiveness is determined on the basis of lack of cross-reactivity between antibodies to one AAV as compared to another AAV. Cross-reactivity differences are usually due to differences in capsid protein sequences/antigenic determinants (e.g., due to VP1, VP2, and/or VP3 sequence differences of AAV serotypes).

Under the traditional definition, a serotype means that the virus of interest has been tested against serum specific for all existing and characterized serotypes for neutralizing activity and no antibodies have been found that neutralize the virus of interest. As more naturally occurring virus isolates are discovered and/or capsid mutants generated, there may or may not be serological differences with any of the currently existing serotypes. Thus, in cases where the new virus (e.g., AAV) has no serological difference, this new virus (e.g., AAV) would be a subgroup or variant of the corresponding serotype. In many cases, serology testing for neutralizing activity has yet to be performed on mutant viruses with capsid sequence modifications to determine if they are of another serotype according to the traditional definition of serotype. Accordingly, for the sake of convenience and to avoid repetition, the term “serotype” broadly refers to both serologically distinct viruses (e.g., AAV) as well as viruses (e.g., AAV) that are not serologically distinct that may be within a subgroup or a variant of a given serotype.

rAAV vectors/particles include any viral strain or serotype. For example and without limitation, a rAAV vector genome or particle (capsid, such as VP1, VP2 and/or VP3) can be based upon any AAV serotype, such as AAV-1, -2, -3, -4, -5, -6, -7, -8, -9, -10, -11, -12, -rh74, -rh10 or AAV-2i8, for example. Such rAAV vectors/particles can be based on the same strain or serotype (or subgroup or variant) or be different from each other. For example and without limitation, a rAAV vector genome or particle (capsid) based upon one serotype genome can be identical to one or more of the capsid proteins that package the vector. In addition, a rAAV vector genome can be based upon an AAV serotype genome distinct from one or more of the capsid proteins that package the vector genome, in which case at least one of the three capsid proteins could be a different AAV serotype, e.g., AAV1, AAV2, AAV3, AAV3B, AAV4, AAV5, AAV6, AAV7, AAV8, AAV9, AAV10, AAV11, AAV12, -rh74, -rh10, AAV-2i8, SPK1 (SEQ ID NO:1), SPK2 (SEQ ID NO:2), or variant thereof, for example. More specifically, a rAAV2 vector genome can comprise AAV2 ITRs but capsids from a different serotype, such as AAV1, AAV3, AAV3B, AAV4, AAV5, AAV6, AAV7, AAV8, AAV9, AAV10, AAV11, AAV12, -rh74, -rh10, AAV-2i8, SPK1 (SEQ ID NO:1), SPK2 (SEQ ID NO:2), or variant thereof, for example. Accordingly, rAAV vectors include gene/protein sequences identical to gene/protein sequences characteristic for a particular serotype, as well as “mixed” serotypes, which also can be referred to as “pseudotypes.”

In certain embodiments, a rAAV vector includes or consists of a capsid sequence at least 70% or more (e.g., 75%, 80%, 85%, 90%, 95%, 96%, 97%, 98%, 99%, 99.5%, etc.) identical to one or more AAV1, AAV2, AAV3, AAV3B, AAV4, AAV5, AAV6, AAV7, AAV8, AAV9, AAV10, AAV11, AAV12, -rh74, -rh10, AAV-2i8, SPK1 (SEQ ID NO:1), or SPK2 (SEQ ID NO:2) capsid proteins (VP1, VP2, and/or VP3 sequences). In certain embodiments, a rAAV vector includes or consists of a sequence at least 70% or more (e.g., 75%, 80%, 85%, 90%, 95%, 96%, 97%, 98%, 99%, 99.5%, etc.) identical to one or more AAV1, AAV2, AAV3, AAV3B, AAV4, AAV5, AAV6, AAV7, AAV8, AAV9, AAV10, AAV11, AAV12, -rh74 or -rh10 ITR(s).

In certain embodiments, rAAV vectors/particles include AAV1, AAV2, AAV3, AAV3B, AAV4, AAV5, AAV6, AAV7, AAV8, AAV9, AAV10, AAV11, AAV12, Rh10, Rh74 and AAV-2i8 variants (e.g., ITR and capsid variants, such as amino acid insertions, additions, substitutions and deletions) thereof, for example, as set forth in WO 2013/158879 (International Application PCT/US2013/037170), WO 2015/013313 (International Application PCT/US2014/047670) and US 2013/0059732 (U.S. application Ser. No. 13/594,773).

rAAV particles, such as AAV1, AAV2, AAV3, AAV3B, AAV4, AAV5, AAV6, AAV7, AAV8, AAV9, AAV10, AAV11, AAV12, -rh74, -rh10, AAV-2i8, SPK1 (SEQ ID NO:1), SPK2 (SEQ ID NO:2) and variants, hybrids and chimeric sequences, can be constructed using recombinant techniques that are known to a skilled artisan, to include one or more heterologous polynucleotide sequences (transgenes) flanked with one or more functional AAV ITR sequences at the 5′ and/or 3′ end. rAAV vectors typically retain at least one functional flanking ITR sequence(s), as necessary for the rescue, replication, and packaging of the recombinant vector into a rAAV vector particle. A rAAV vector genome would therefore include sequences required in cis for replication and packaging (e.g., functional ITR sequences).

Host cells for producing recombinant AAV particles include but are not limited to microorganisms, yeast cells, insect cells, and mammalian cells that can be, or have been, used as recipients of a heterologous rAAV vectors. Cells from the stable human cell line, HEK293 (readily available through, e.g., the American Type Culture Collection under Accession Number ATCC CRL1573) can be used. In certain embodiments, a modified human embryonic kidney cell line (e.g., HEK293), which is transformed with adenovirus type-5 DNA fragments, and expresses the adenoviral E1a and E1b genes, is used to generate recombinant AAV particles. The modified HEK293 cell line is readily transfected, and provides a particularly convenient platform in which to produce rAAV particles. Other host cell lines appropriate for recombinant AAV production are described in International Application PCT/2017/024951.

In certain embodiments, AAV helper functions are introduced into the host cell by transfecting the host cell with an AAV helper construct either prior to, or concurrently with, the transfection of an AAV expression vector. AAV helper constructs are thus sometimes used to provide at least transient expression of AAV rep and/or cap genes to complement missing AAV functions necessary for productive AAV transduction. AAV helper constructs often lack AAV ITRs and can neither replicate nor package themselves. These constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products. A number of other vectors are known which encode Rep and/or Cap expression products.

Methods of generating recombinant AAV vectors/particles capable of transducing mammalian cells are known in the art. For example, recombinant AAV vectors/particles can be produced as described in U.S. Pat. No. 9,408,904; and International Applications PCT/US2017/025396 and PCT/US2016/064414.

The terms “nucleic acid” and “polynucleotide” are used interchangeably herein to refer to all forms of nucleic acid, oligonucleotides, including deoxyribonucleic acid (DNA) and ribonucleic acid (RNA). Nucleic acids include genomic DNA, cDNA and antisense DNA, and spliced or unspliced mRNA, rRNA tRNA and inhibitory DNA or RNA (RNAi, e.g., small or short hairpin (sh)RNA, microRNA (miRNA), small or short interfering (si)RNA, trans-splicing RNA, or antisense RNA). Nucleic acids include naturally occurring, synthetic, and intentionally modified or altered polynucleotides (e.g., variant nucleic acid).

Nucleic acids such as vector genome, cDNA, genomic DNA, RNA, and fragments thereof can be single, double, or triplex, linear or circular, and can be of any length. In discussing nucleic acids, a sequence or structure of a particular nucleic acid may be described herein according to the convention of providing the sequence in the 5′ to 3′ direction.

A “transgene” is used herein to conveniently refer to a heterologous nucleic acid that is intended or has been introduced into a cell or organism. Transgenes include any heterologous nucleic acid, such as a nucleic acid encoding TPP1.

The term “transduce” and grammatical variations thereof refer to introduction of a molecule such as an rAAV vector into a cell or host organism. The heterologous nucleic acid/transgene may or may not be integrated into genomic nucleic acid of the recipient cell. The introduced heterologous nucleic acid may also exist in the recipient cell or host organism extrachromosomally, or only transiently.

A “transduced cell” is a cell into which the transgene has been introduced. Accordingly, a “transduced” cell (e.g., in a mammal, such as a cell or tissue or organ cell), means a genetic change in a cell following incorporation, for example, of a nucleic acid (e.g., a transgene) into the cell. Thus, a “transduced” cell is a cell into which, or a progeny thereof in which an exogenous nucleic acid (e.g., nucleic acid encoding TPP1) has been introduced. The cell(s) can be propagated and the introduced protein expressed. For gene therapy uses and methods, a transduced cell can be in a subject, such as a mammal, a primate, or a human.

An “expression control element” refers to nucleic acid sequence(s) that influence expression of an operably linked nucleic acid. Expression control elements as set forth herein include promoters and enhancers. Vector sequences including AAV vectors can include one or more “expression control elements.” Typically, such elements are included to facilitate proper heterologous polynucleotide transcription and as appropriate translation (e.g., a promoter, enhancer, splicing signal for introns, maintenance of the correct reading frame of the gene to permit in-frame translation of mRNA and, stop codons etc.). Such elements typically act in cis, referred to as a “cis acting” element, but may also act in trans.

Expression control can be effected at the level of transcription, translation, splicing, message stability, etc. Typically, an expression control element that modulates transcription is juxtaposed near the 5′ end (i.e., “upstream”) of a transcribed nucleic acid. Expression control elements can also be located at the 3′ end (i.e., “downstream”) of the transcribed sequence or within the transcript (e.g., in an intron). Expression control elements can be located adjacent to or at a distance away from the transcribed sequence (e.g., 1-10, 10-25, 25-50, 50-100, 100 to 500, or more nucleotides from the polynucleotide), even at considerable distances. Nevertheless, owing to the length limitations of AAV vectors, expression control elements will typically be within 1 to 1000 nucleotides from the transcription start site of the heterologous nucleic acid.

Functionally, expression of operably linked nucleic acid is at least in part controllable by the element (e.g., promoter) such that the element modulates transcription of the nucleic acid and, as appropriate, translation of the transcript. A specific example of an expression control element is a promoter, which is usually located 5′ of the transcribed nucleic acid sequence. A promoter typically increases an amount expressed from operably linked nucleic acid as compared to an amount expressed when no promoter exists.

An “enhancer” as used herein can refer to a sequence that is located adjacent to the heterologous nucleic acid Enhancer elements are typically located upstream of a promoter element but also function and can be located downstream of or within a sequence. Hence, an enhancer element can be located 10-50 base pairs, 50-100 base pairs, 100-200 base pairs, or 200-300 base pairs, or more base pairs upstream or downstream of a heterologous nucleic acid sequence Enhancer elements typically increase expressed of an operably linked nucleic acid above expression afforded by a promoter element.

An expression construct or cassette may comprise regulatory elements which serve to drive expression in a particular cell or tissue type. Expression control elements (e.g., promoters) include those active in a particular tissue or cell type, referred to herein as a “tissue-specific expression control elements/promoters.” Tissue-specific expression control elements are typically active in specific cell or tissue (e.g., liver). Expression control elements are typically active in particular cells, tissues or organs because they are recognized by transcriptional activator proteins, or other regulators of transcription, that are unique to a specific cell, tissue or organ type. Such regulatory elements are known to those of skill in the art (see, e.g., Sambrook et al. (1989) and Ausubel et al. (1992)).

Expression control elements also include ubiquitous or promiscuous promoters/enhancers which are capable of driving expression of a polynucleotide in many different cell types. Such elements include, but are not limited to the cytomegalovirus (CMV) immediate early promoter/enhancer sequences, the Rous sarcoma virus (RSV) promoter/enhancer sequences and the other viral promoters/enhancers active in a variety of mammalian cell types, or synthetic elements that are not present in nature (see, e.g., Boshart et al., Cell, 41:521-530 (1985)), the SV40 promoter, the dihydrofolate reductase promoter, the cytoplasmic β-actin promoter and the phosphoglycerol kinase (PGK) promoter.

Expression control elements also can confer expression in a manner that is regulatable, that is, a signal or stimuli increases or decreases expression of the operably linked heterologous polynucleotide. A regulatable element that increases expression of the operably linked polynucleotide in response to a signal or stimuli is also referred to as an “inducible element” (i.e., is induced by a signal). Particular examples include, but are not limited to, a hormone (e.g., steroid) inducible promoter. Typically, the amount of increase or decrease conferred by such elements is proportional to the amount of signal or stimuli present; the greater the amount of signal or stimuli, the greater the increase or decrease in expression. Regulatable expression control elements include, for example and without limitation, the zinc-inducible sheep metallothionine (MT) promoter; the steroid hormone-inducible mouse mammary tumor virus (MMTV) promoter; the T7 polymerase promoter system (WO 98/10088); the tetracycline-repressible system (Gossen, et al., Proc. Natl. Acad. Sci. USA, 89:5547-5551 (1992)); the tetracycline-inducible system (Gossen, et al., Science. 268:1766-1769 (1995); see also Harvey, et al., Curr. Opin. Chem. Biol. 2:512-518 (1998)); the RU486-inducible system (Wang, et al., Nat. Biotech. 15:239-243 (1997) and Wang, et al., Gene Ther. 4:432-441 (1997)]; and the rapamycin-inducible system (Magari, et al., J. Clin. Invest. 100:2865-2872 (1997); Rivera, et al., Nat. Medicine. 2:1028-1032 (1996)). Other regulatable control elements which may be used in the invention are those which are regulated by a specific physiological state, e.g., temperature, acute phase, development.

Expression control elements also include the native elements(s) for the heterologous polynucleotide. A native control element (e.g., promoter) may be used in the invention when it is desired that expression of the heterologous polynucleotide should mimic the native expression. The native element may be used in the invention when expression of the heterologous polynucleotide is to be regulated temporally or developmentally, or in a tissue-specific manner, or in response to specific transcriptional stimuli. Other native expression control elements, such as introns, polyadenylation sites or Kozak consensus sequences may also be used.

The term “operably linked” means that the regulatory sequences necessary for expression of a nucleic acid sequence are placed in the appropriate positions relative to the sequence so as to effect expression of the nucleic acid sequence. This same definition is sometimes applied to the arrangement of nucleic acid sequences and transcription control elements (e.g., promoters, enhancers, and termination elements) in an expression vector, e.g., rAAV vector.

In the example of an expression control element in operable linkage with a nucleic acid, the relationship is such that the control element modulates expression of the nucleic acid. More specifically, for example and without limitation, two DNA sequences operably linked means that the two DNAs are arranged (cis or trans) in such a relationship that at least one of the DNA sequences is able to exert a physiological effect upon the other sequence.

Accordingly, additional elements for vectors include, without limitation, an expression control (e.g., promoter/enhancer) element, a transcription termination signal or stop codon, 5′ or 3′ untranslated regions (e.g., polyadenylation (polyA) sequences) which flank a sequence, such as one or more copies of an AAV ITR sequence, or an intron.

Further elements include, for example and without limitation, filler or stuffer polynucleotide sequences, for example to improve packaging and reduce the presence of contaminating nucleic acid. AAV vectors typically accept inserts of DNA having a size range which is generally about 4 kb to about 5.2 kb, or slightly more. Thus, for shorter sequences, inclusion of a stuffer or filler in order to adjust the length to near or at the normal size of the virus genomic sequence acceptable for AAV vector packaging into virus particle. In certain embodiments, a filler/stuffer nucleic acid sequence is an untranslated (non-protein encoding) segment of nucleic acid. For a nucleic acid sequence less than 4.7 Kb, the filler or stuffer polynucleotide sequence has a length that when combined (e.g., inserted into a vector) with the sequence has a total length between about 3.0-5.5 Kb, or between about 4.0-5.0 Kb, or between about 4.3-4.8 Kb.

The term “isolated,” when used as a modifier of a composition, means that the compositions are made by the hand of man or are separated, completely or at least in part, from their naturally occurring in vivo environment. Generally, isolated compositions are substantially free of one or more materials with which they normally associate with in nature, for example and without limitation, one or more protein, nucleic acid, lipid, carbohydrate, cell membrane.

The term “isolated” does not exclude combinations produced by the hand of man, for example and without limitation, a rAAV sequence, or rAAV particle that packages or encapsidates an AAV vector genome and a pharmaceutical formulation. The term “isolated” also does not exclude alternative physical forms of the composition, such as hybrids/chimeras, multimers/oligomers, modifications (e.g., phosphorylation, glycosylation, lipidation) or derivatized forms, or forms expressed in host cells produced by the hand of man.

The term “substantially pure” refers to a preparation comprising at least 50-60% by weight the compound of interest (e.g., nucleic acid, oligonucleotide, protein, etc.). The preparation can comprise at least 75% by weight, or at least 85% by weight, or about 90-99% by weight, of the compound of interest. Purity is measured by methods appropriate for the compound of interest (e.g. chromatographic methods, agarose or polyacrylamide gel electrophoresis, HPLC analysis, and the like).

The phrase “consisting essentially of” when referring to a particular nucleotide sequence or amino acid sequence means a sequence having the properties of a given SEQ ID NO. For example and without limitation, when used in reference to an amino acid sequence, the phrase includes the sequence per se and molecular modifications that would not affect the basic and novel characteristics of the sequence.

Nucleic acids, expression vectors (e.g., AAV vector genomes), plasmids, including nucleic acids encoding TPP1 may be prepared by using recombinant DNA technology methods. The availability of nucleotide sequence information enables preparation of isolated nucleic acid molecules of the invention by a variety of means. Nucleic acids encoding TPP1 can be made using various standard cloning, recombinant DNA technology, via cell expression or in vitro translation and chemical synthesis techniques. Purity of polynucleotides can be determined through sequencing, gel electrophoresis and the like. For example and without limitation, nucleic acids can be isolated using hybridization or computer-based database screening techniques. Such techniques include, but are not limited to: (1) hybridization of genomic DNA or cDNA libraries with probes to detect homologous nucleotide sequences; (2) antibody screening to detect polypeptides having shared structural features, for example and without limitation, using an expression library; (3) polymerase chain reaction (PCR) on genomic DNA or cDNA using primers capable of annealing to a nucleic acid sequence of interest; (4) computer searches of sequence databases for related sequences; and (5) differential screening of a subtracted nucleic acid library.

Nucleic acids may be maintained as DNA in any convenient cloning vector. In certain embodiments, clones are maintained in a plasmid cloning/expression vector, such as pBluescript (Stratagene, La Jolla, Calif.), which is propagated in a suitable E. coli host cell. Alternatively, nucleic acids may be maintained in vector suitable for expression in mammalian cells, for example and without limitation, an AAV vector. In cases where post-translational modification affects protein function, nucleic acid molecule can be expressed in mammalian cells.

In certain embodiments, rAAV vectors may optionally comprise regulatory elements necessary for expression of the heterologous nucleic acid in a cell positioned in such a manner as to permit expression of the encoded protein in the host cell. Such regulatory elements required for expression include, but are not limited to, promoter sequences, enhancer sequences and transcription initiation sequences as set forth herein and known to the skilled artisan.

Methods and uses of the invention include delivering (transducing) nucleic acid (transgene) into host cells, including dividing and/or non-dividing cells. The nucleic acids, rAAV vector, methods, uses and pharmaceutical formulations of the invention are additionally useful in a method of delivering, administering or providing sequence encoded by heterologous nucleic acid to a subject in need thereof, as a method of treatment. In this manner, the nucleic acid is transcribed and a protein produced in vivo in a subject. The subject may benefit from or be in need of the protein because the subject has a deficiency of the protein, or because production of the protein in the subject may impart some therapeutic effect, as a method of treatment or otherwise.

The invention is useful in animals including human and veterinary medical applications. Suitable subjects therefore include mammals, such as humans, as well as non-human mammals. The term “subject” refers to an animal, typically a mammal, such as humans, non-human primates (apes, gibbons, gorillas, chimpanzees, orangutans, macaques), a domestic animal (dogs and cats) and experimental animals (mouse, rat, rabbit, guinea pig). Human subjects include fetal, neonatal, infant, juvenile and young adult subjects. Subjects include animal disease models, for example and without limitation, mouse and other animal models of protein/enzyme deficiencies such as CLN2.

Subjects appropriate for treatment in accordance with the invention include those having or at risk of having a TPP1 deficiency or insufficiency, or produce an aberrant, partially functional or non-functional TPP1. Subjects can be tested for TPP1 expression and/or activity to determine if such subjects are appropriate for treatment according to methods of the invention. Subjects can also be tested for mutation in the endogenous nucleic acid encoding TPP1. Certain genetic mutations are known to reduce or destroy TPP1 activity. Subjects appropriate for treatment in accordance with the invention also include those subjects that would benefit from TPP1. Treated subjects can be monitored after treatment periodically, e.g., every 1-4 weeks, 1-6 months, 6-12 months, or 1, 2, 3, 4, 5 or more years.

Assays to detect and/or measure TPP1 activity are known in the art, including assays described in Liu et al., 2017, Clin. Chem., 63:1118-1126, doi:10.1373/clinchem.2016.269167 and Barcenas et al., 2014, Anal. Chem., 87:7962-7968.

Subjects can be tested for an immune response, e.g., antibodies against AAV. Candidate subjects can therefore be screened prior to treatment according to a method of the invention. Subjects also can be tested for antibodies against AAV after treatment, and optionally monitored for a period of time after treatment. Subjects developing AAV antibodies can be treated with an immunosuppressive agent, or other regimen as set forth herein.

Subjects appropriate for treatment in accordance with the invention also include those having or at risk of producing antibodies against AAV (anti-AAV antibodies). rAAV vectors can be administered or delivered to such subjects using several techniques. For example and without limitation, AAV empty capsid (i.e., AAV lacking vector genome) can be delivered to bind to the anti-AAV antibodies in the subject thereby allowing the rAAV vector comprising the heterologous nucleic acid to transduce cells of the subject.

As set forth herein, rAAV are useful as gene therapy vectors as they can penetrate cells and introduce nucleic acid/genetic material into the cells. Because AAV are not associated with pathogenic disease in humans, rAAV vectors are able to deliver heterologous polynucleotide sequences (e.g., therapeutic proteins and agents) to human patients without causing substantial AAV pathogenesis or disease.

rAAV vectors possess a number of desirable features for such applications, including tropism for dividing and non-dividing cells. Early clinical experience with these vectors also demonstrated no sustained toxicity and immune responses are typically minimal or undetectable. AAV are known to infect a wide variety of cell types in vivo by receptor-mediated endocytosis or by transcytosis. These vector systems have been tested in humans targeting many tissues, such as central nervous system, brain, retinal epithelium, liver, skeletal muscle, airways, joints and hematopoietic stem cells.

It may be desirable to introduce a rAAV vector that can provide, for example and without limitation, multiple copies of TPP1 and hence greater amounts of TPP1 protein. Improved rAAV vectors and methods for producing these vectors have been described in detail in a number of references, patents, and patent applications, including: Wright J. F. (Hum Gene Ther 20:698-706, 2009).

rAAV vectors may be administered to a patient via infusion in a biologically compatible carrier, for example and without limitation, via intracranial injection. rAAV vectors may be administered alone or in combination with other molecules. Accordingly, rAAV vectors and other compositions, agents, drugs, biologics (proteins) can be incorporated into pharmaceutical compositions. Such pharmaceutical compositions are useful for, among other things, administration and delivery to a subject in vivo or ex vivo.

In certain embodiments, pharmaceutical compositions also contain a pharmaceutically or biologically acceptable carrier or excipient. Such excipients include any pharmaceutical agent that does not itself induce an immune response harmful to the individual receiving the composition, and which may be administered without undue toxicity.

As used herein the term “pharmaceutically acceptable” and “physiologically acceptable” mean a biologically acceptable formulation, gaseous, liquid or solid, or mixture thereof, which is suitable for one or more routes of administration, in vivo delivery or contact. A “pharmaceutically acceptable” or “physiologically acceptable” composition is a material that is not biologically or otherwise undesirable, e.g., the material may be administered to a subject without causing substantial undesirable biological effects. Thus, such a pharmaceutical composition may be used in the invention, for example, in administering a nucleic acid, vector, viral particle or protein to a subject.

Pharmaceutically acceptable excipients include, but are not limited to, liquids such as water, saline, glycerol, sugars and ethanol. Pharmaceutically acceptable salts can also be included therein, for example and without limitation, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles.

The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding, free base forms. In other cases, a preparation may be a lyophilized powder which may contain any or all of the following: 1-50 mM histidine, 0.1%-2% sucrose, and 2-7% mannitol, at a pH range of 4.5 to 5.5, that is combined with buffer prior to use.

Pharmaceutical compositions can be formulated to be compatible with a particular route of administration or delivery, as set forth herein or known to one of skill in the art. Thus, pharmaceutical compositions include carriers, diluents, or excipients suitable for administration by various routes.

Compositions suitable for parenteral administration comprise aqueous and non-aqueous solutions, suspensions or emulsions of the active compound, which preparations are typically sterile and can be isotonic with the blood of the intended recipient. Compositions comprise, for example and without limitation water, buffered saline, Hanks' solution, Ringer's solution, dextrose, fructose, ethanol, animal, vegetable or synthetic oils. Aqueous injection suspensions may contain substances which increase the viscosity of the suspension, such as sodium carboxymethyl cellulose, sorbitol, or dextran.

Additionally, suspensions of the active compounds may be prepared as appropriate oil injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

Cosolvents and adjuvants may be added to the formulation. Cosolvents may contain hydroxyl groups or other polar groups, for example and without limitation, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example and without limitation, surfactants such as, soya lecithin and oleic acid; sorbitan esters such as sorbitan trioleate; and polyvinylpyrrolidone.

After pharmaceutical compositions have been prepared, they may be placed in an appropriate container and labeled for treatment. Such labeling could include amount, frequency, and method of administration.

Pharmaceutical compositions and delivery systems appropriate for the compositions, methods and uses of the invention are known in the art (see, e.g., Remington: The Science and Practice of Pharmacy (2003) 20^{th} ed., Mack Publishing Co., Easton, Pa.; Remington's Pharmaceutical Sciences (1990) 18^{th} ed., Mack Publishing Co., Easton, Pa.; The Merck Index (1996) 12^{th} ed., Merck Publishing Group, Whitehouse, N.J.; Pharmaceutical Principles of Solid Dosage Forms (1993), Technomic Publishing Co., Inc., Lancaster, Pa.; Ansel and Stoklosa, Pharmaceutical Calculations (2001) 11^{th} ed., Lippincott Williams & Wilkins, Baltimore, Md.; and Poznansky et al., Drug Delivery Systems (1980), R. L. Juliano, ed., Oxford, N.Y., pp. 253-315).

An “effective amount” or “sufficient amount” refers to an amount that provides, in single or multiple doses, alone or in combination, with one or more other compositions (therapeutic or immunosuppressive agents such as a drug), treatments, protocols, or therapeutic regimens agents, a detectable response of any duration of time (long or short term), an expected or desired outcome in or a benefit to a subject of any measurable or detectable degree or for any duration of time (e.g., for minutes, hours, days, months, years, or cured).

Doses can vary and depend upon the type, onset, progression, severity, frequency, duration, or probability of the disease to which treatment is directed, the clinical endpoint desired, previous or simultaneous treatments, the general health, age, gender, race or immunological competency of the subject and other factors that will be appreciated by the skilled artisan. The dose amount, number, frequency or duration may be proportionally increased or reduced, as indicated by any adverse side effects, complications or other risk factors of the treatment or therapy and the status of the subject. The skilled artisan will appreciate the factors that may influence the dosage and timing required to provide an amount sufficient for providing a therapeutic or prophylactic benefit.

The dose to achieve a therapeutic effect, e.g., the dose in vector genomes per kilogram of body weight (vg/kg) of the subject or patient, or the dose in vector genomes per brain (vg/brain) of the subject or patient, or the dose in vector genomes delivered to the CNS (vg/CNS) of the subject or patient, will vary based on several factors including, but not limited to: route of administration, the level of heterologous polynucleotide expression required to achieve a therapeutic effect, the specific disease treated, any host immune response to the viral vector, a host immune response to the heterologous polynucleotide or expression product (protein), and the stability of the protein expressed.

Generally, doses will be greater than about 1.5×10¹³ recombinant AAV vector genomes. For example, a dose of about 5×10¹³ recombinant AAV vector genomes or greater than about 5×10¹³ recombinant AAV vector genomes; a dose of about 1×10¹⁴ recombinant AAV vector genomes or greater than about 1×10¹⁴ recombinant AAV vector genomes; a dose of about 5×10¹⁴ recombinant AAV vector genomes or greater than about 5×10¹⁴ recombinant AAV vector genomes; a dose of about 1×10¹⁵ recombinant AAV vector genomes or greater than about 1×10¹⁵ recombinant AAV vector genomes; and a dose of about 5×10¹⁵ recombinant AAV vector genomes or greater than about 5×10¹⁵ recombinant AAV vector genomes.

In certain embodiments, recombinant AAV vector genomes are administered at a dose range from about 1.5×10¹³ to about 5×10¹⁵ recombinant AAV vector genomes; a dose range from about 1×10¹⁴ to about 3×10¹⁵ recombinant AAV vector genomes; a dose range from about 2×10¹⁴ to about 2×10¹⁵ recombinant AAV vector genomes; a dose range from about 2.5×10¹⁴ to about 7.5×10¹⁴ recombinant AAV vector genomes; a dose range from about 5×10¹⁴ to about 5×10¹⁵ recombinant AAV vector genomes; and a dose range from about 1×10¹⁵ to about 5×10¹⁵ recombinant AAV vector genomes.

In certain embodiments, rAAV vector genomes are administered at a dose of about 1×10¹⁴ vector genomes, administered at a dose of about 2×10¹⁴ vector genomes, administered at a dose of about 3×10¹⁴ vector genomes, administered at a dose of about 4×10¹⁴ vector genomes, administered at a dose of about 5×10¹⁴ vector genomes, administered at a dose of about 6×10¹⁴ vector genomes, administered at a dose of about 7×10¹⁴ vector genomes, administered at a dose of about 8×10¹⁴ vector genomes, administered at a dose of about 9×10¹⁴ vector genomes, administered at a dose of about 1×10¹⁵ vector genomes, administered at a dose of about 2×10¹⁵ vector genomes, administered at a dose of about 3×10¹⁵ vector genomes, administered at a dose of about 4×10¹⁵ vector genomes, or administered at a dose of about 5×10¹⁵ vector genomes.

In certain embodiments, doses will be greater than about 1.5×10¹³ rAAV vg/brain of the subject or patient. For example, a dose of about 5×10¹³ rAAV vg/brain or greater than about 5×10¹³ rAAV vg/brain; a dose of about 1×10¹⁴ rAAV vg/brain or greater than about 1×10¹⁴ rAAV vg/brain; a dose of about 5×10¹⁴ rAAV vg/brain or greater than about 5×10¹⁴ rAAV vg/brain; a dose of about 1×10¹⁵ rAAV vg/brain or greater than about 1×10¹⁵ rAAV vg/brain; and a dose of about 5×10¹⁵ rAAV vg/brain or greater than about 5×10¹⁵ rAAV vg/brain.

In certain embodiments, rAAV vg are administered at a dose range from about 1.5×10¹³ to about 5×10¹⁵ rAAV vg/brain; a dose range from about 1×10¹⁴ to about 3×10¹⁵ rAAV vg/brain; a dose range from about 2×10¹⁴ to about 2×10¹⁵ rAAV vg/brain; a dose range from about 2.5×10¹⁴ to about 7.5×10¹⁴ rAAV vg/brain; a dose range from about 5×10¹⁴ to about 5×10¹⁵ rAAV vg/brain; and a dose range from about 1×10¹⁵ to about 5×10¹⁵ rAAV vg/brain.

In certain embodiments, rAAV vg are administered at a dose of about 1×10¹⁴ rAAV vg/brain, administered at a dose of about 2×10¹⁴ rAAV vg/brain, administered at a dose of about 3×10¹⁴ rAAV vg/brain, administered at a dose of about 4×10¹⁴ rAAV vg/brain, administered at a dose of about 5×10¹⁴ rAAV vg/brain, administered at a dose of about 6×10¹⁴ rAAV vg/brain, administered at a dose of about 7×10¹⁴ rAAV vg/brain, administered at a dose of about 8×10¹⁴ rAAV vg/brain, administered at a dose of about 9×10¹⁴ rAAV vg/brain, administered at a dose of about 1×10¹⁵ rAAV vg/brain, administered at a dose of about 2×10¹⁵ rAAV vg/brain, administered at a dose of about 3×10¹⁵ rAAV vg/brain, administered at a dose of about 4×10¹⁵ rAAV vg/brain, or administered at a dose of about 5×10¹⁵ rAAV vg/brain.

A “unit dosage form” as used herein refers to physically discrete units suited as unitary dosages for the subject to be treated; each unit containing a predetermined quantity optionally in association with a pharmaceutical carrier (excipient, diluent, vehicle or filling agent) which, when administered in one or more doses, is calculated to produce a desired effect (e.g., prophylactic or therapeutic effect). Unit dosage forms may be within, for example, ampules and vials, which may include a liquid composition, or a composition in a freeze-dried or lyophilized state; a sterile liquid carrier, for example, can be added prior to administration or delivery in vivo. Individual unit dosage forms can be included in multi-dose kits or containers. rAAV particles, and pharmaceutical compositions thereof can be packaged in single or multiple unit dosage form for ease of administration and uniformity of dosage.

The doses of an “effective amount” or “sufficient amount” for treatment (e.g., to ameliorate or to provide a therapeutic benefit or improvement) typically are effective to provide a response to one, multiple or all adverse symptoms, consequences or complications of the disease, one or more adverse symptoms, disorders, illnesses, pathologies, or complications, for example, caused by or associated with the disease, to a measurable extent, although decreasing, reducing, inhibiting, suppressing, limiting or controlling progression or worsening of the disease is a satisfactory outcome.

An effective amount or a sufficient amount can but need not be provided in a single administration, may require multiple administrations, and, can but need not be, administered alone or in combination with another composition (e.g., agent), treatment, protocol or therapeutic regimen. For example, the amount may be proportionally increased as indicated by the need of the subject, type, status and severity of the disease treated or side effects (if any) of treatment. In addition, an effective amount or a sufficient amount need not be effective or sufficient if given in single or multiple doses without a second composition (e.g., another drug or agent), treatment, protocol or therapeutic regimen, since additional doses, amounts or duration above and beyond such doses, or additional compositions (e.g., drugs or agents), treatments, protocols or therapeutic regimens may be included in order to be considered effective or sufficient in a given subject. Amounts considered effective also include amounts that result in a reduction of the use of another treatment, therapeutic regimen or protocol, such as administration of nucleic acid encoding TPP1 for treatment of a TPP1 deficiency (e.g., CLN2).

Accordingly, methods and uses of the invention also include, among other things, methods and uses that result in a reduced need or use of another compound, agent, drug, therapeutic regimen, treatment protocol, process, or remedy. Thus, in accordance with the invention, methods and uses of reducing need or use of another treatment or therapy are provided.

An effective amount or a sufficient amount need not be effective in each and every subject treated, nor a majority of treated subjects in a given group or population. An effective amount or a sufficient amount means effectiveness or sufficiency in a particular subject, not a group or the general population. As is typical for such methods, some subjects will exhibit a greater response, or less or no response to a given treatment method or use.

Administration or in vivo delivery to a subject can be performed prior to development of an adverse symptom, condition, complication, etc. caused by or associated with the disease. For example, a screen (e.g., genetic) can be used to identify such subjects as candidates for invention compositions, methods and uses. Such subjects therefore include those screened positive for an insufficient amount or a deficiency in a functional gene product (e.g., TPP1 deficiency), or that produce an aberrant, partially functional or non-functional gene product (e.g., TPP1).

Administration or in vivo delivery to a subject in accordance with the methods and uses of the invention as disclosed herein can be practiced within 1-2, 2-4, 4-12, 12-24 or 24-72 hours after a subject has been identified as having the disease targeted for treatment, has one or more symptoms of the disease, or has been screened and is identified as positive as set forth herein even though the subject does not have one or more symptoms of the disease. Of course, methods and uses of the invention can be practiced 1-7, 7-14, 14-24, 24-48, 48-64 or more days, months or years after a subject has been identified as having the disease targeted for treatment, has one or more symptoms of the disease, or has been screened and is identified as positive as set forth herein.

The term “ameliorate” means a detectable or measurable improvement in a subject's disease or symptom thereof, or an underlying cellular response. A detectable or measurable improvement includes a subjective or objective decrease, reduction, inhibition, suppression, limit or control in the occurrence, frequency, severity, progression, or duration of the disease, or complication caused by or associated with the disease, or an improvement in a symptom or an underlying cause or a consequence of the disease, or a reversal of the disease.

For CLN2, an effective amount would be an amount that inhibits, reduces, or ameliorates vision impairment, impaired or stunted cognitive development, loss of motor control or seizures. An effective amount also would be an amount that stabilizes or inhibits or prevents worsening of an adverse symptom of CLN2.

Therapeutic doses will depend on, among other factors, the age and general condition of the subject, the severity of the disease or disorder. A therapeutically effective amount in humans will fall in a relatively broad range that may be determined by a medical practitioner based on the response of an individual patient.

Compositions such as pharmaceutical compositions may be delivered to a subject, so as to allow production of the encoded protein. In certain embodiments, pharmaceutical compositions comprise sufficient genetic material to enable a recipient to produce a therapeutically effective amount of a protein in the subject.

Compositions may be formulated and/or administered in any sterile, biocompatible pharmaceutical carrier, including, but not limited to, saline, buffered saline, dextrose, and water. The compositions may be formulated and/or administered to a patient alone, or in combination with other agents (e.g., co-factors) which influence hemostasis.

Methods of treatment of the invention include delivery and administration systemically, regionally or locally, or by any route, for example, by injection or infusion. Delivery of the pharmaceutical compositions in vivo may generally be accomplished via injection. For example, rAAV vectors/particles may be administered intracranially, for example, into the CNS, in particular, for example, into a portion of the brain such as a lateral ventricle.

Methods of treatment and rAAV vectors according to the invention include combination therapies that include the additional use of any compound, agent, drug, treatment or other therapeutic regimen or protocol having a desired therapeutic, beneficial, additive, synergistic or complementary activity or effect. Exemplary combination compositions and treatments include second actives, such as, biologics (proteins), agents (e.g., immunosuppressive agents) and drugs. Such biologics (proteins), agents, drugs, treatments and therapies can be administered or performed prior to, substantially contemporaneously with or following any other method or treatment according to the invention.

The compound, agent, drug, treatment or other therapeutic regimen or protocol can be administered as a combination composition, or administered separately, such as concurrently or in series or sequentially (prior to or following) delivery or administration of a nucleic acid, vector, or rAAV particle. The invention therefore provides combinations in which a method of treatment according to the invention is in a combination with any compound, agent, drug, therapeutic regimen, treatment protocol, process, remedy or composition, set forth herein or known to one of skill in the art. The compound, agent, drug, therapeutic regimen, treatment protocol, process, remedy or composition can be administered or performed prior to, substantially contemporaneously with or following administration of a nucleic acid, vector or rAAV particle administered to a patient according to the invention.

In certain embodiments, at least one an immunosuppressive agent is administered to a subject prior to, substantially contemporaneously with or after administration of a rAAV vector to the subject. In certain embodiments, an immunosuppressive agent is anti-inflammatory agent. In certain embodiments, an immunosuppressive agent is a steroid. In certain embodiments, an immunosuppressive agent is prednisone, cyclosporine (e.g., cyclosporine A), mycophenolate, rituximab, or a derivative thereof.

Strategies to reduce (overcome) or avoid humoral immunity to AAV in gene transfer include, administering high vector doses, use of AAV empty capsids as decoys to adsorb anti-AAV antibodies, administration of immunosuppressive drugs to decrease, reduce, inhibit, prevent or eradicate the humoral immune response to AAV, changing the AAV capsid serotype or engineering the AAV capsid to be less susceptible to neutralizing antibodies, use of plasma exchange cycles to adsorb anti-AAV immunoglobulins, thereby reducing anti-AAV antibody titer, and use of delivery techniques such as balloon catheters followed by saline flushing. Such strategies are described in Mingozzi et al., 2013, Blood, 122:23-36.

Exemplary ratio of AAV empty capsids to the rAAV vector can be within or between about 100:1-50:1, from about 50:1-25:1, from about 25:1-10:1, from about 10:1-1:1, from about 1:1-1:10, from about 1:10-1:25, from about 1:25-1:50, or from about 1:50-1:100. Ratios can also be about 2:1, 3:1, 4:1, 5:1, 6:1, 7:1, 8:1, 9:1, or 10:1.

Amounts of AAV empty capsids to administer can be calibrated based upon the amount (titer) of AAV antibodies produced in a particular subject.

AAV antibodies may be preexisting and may be present at levels that reduce or block TPP1 gene transfer vector transduction of target cells. Alternatively, AAV antibodies may develop after exposure to AAV or administration of an AAV vector. If such antibodies develop after administration of an AAV vector, these subjects can also be treated accordingly.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All patents, patent applications, publications, and other references, GenBank citations and ATCC citations cited herein are incorporated by reference in their entirety. In case of conflict, the specification, including definitions, will control.

All of the features disclosed herein may be combined in any combination. Each feature disclosed in the specification may be replaced by an alternative feature serving a same, equivalent, or similar purpose. Thus, unless expressly stated otherwise, disclosed features (e.g., nucleic acid encoding TPP1, expression cassettes comprising a nucleic acids encoding TPP1, and rAAV particles comprising the nucleic acid encoding TPP1) are an example of a genus of equivalent or similar features.

As used herein, the singular forms “a”, “and,” and “the” include plural referents unless the context clearly indicates otherwise. Thus, for example, reference to “a nucleic acid” includes a plurality of such nucleic acids, reference to “a vector” includes a plurality of such vectors, and reference to “a virus” or “particle” includes a plurality of such viruses/particles.

As used herein, all numerical values or numerical ranges include integers within such ranges and fractions of the values or the integers within ranges unless the context clearly indicates otherwise. Thus, to illustrate, reference to 86% or more identity, includes 86%, 87%, 88%, 89%, 90%, 91%, 92%, 93%, 94%, 95%, 96%, 97%, 98%, 99%, 100%, etc., as well as 86.1%, 86.2%, 86.3%, 86.4%, 86.5%, etc., 87.1%, 88.2%, 88.3%, 88.4%, 88.5%, etc., and so forth.

Reference to an integer with more (greater) or less than includes any number greater or less than the reference number, respectively. Thus, for example, a reference to greater than 1.5×10¹³, includes 1.6×10¹³, 1.7×10¹³, 1.8×10¹³, 1.9×10¹³, 2×10¹³, 2.1×10¹³, 2.2×10¹³, 2.3×10¹³, 2.4×10¹³, 2.5×10¹³, 2.6×10¹³, 2.7×10¹³, 2.8×10¹³, 2.9×10¹³, 3×10¹³, 3.1×10¹³, 3.2×10¹³, etc.

As used herein, all numerical values or ranges include sub ranges and fractions of the values and integers within such ranges and sub ranges and the wrong 1 as well as the file okay thanks fractions of the integers within such ranges unless the context clearly indicates otherwise. Thus, to illustrate, reference to a numerical range, such as 1-10 includes 1-2, 1-3, 1-4, 1-5, 1-6, 1-7, 1-8, 1-9, 2-3, 2-4, 2-5, 2-6, 2-7, 2-8, 2-9, 2-10, 3-4, 3-5, 3-6, 3-7, 3-8, 3-9, 3-10, etc.; and 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-850, includes ranges of 1-20, 1-30, 1-40, 1-50, 1-60, 10-30, 10-40, 10-50, 10-60, 10-70, 10-80, 20-40, 20-50, 20-60, 20-70, 20-80, 20-90, 50-75, 50-100, 50-150, 50-200, 50-250, 100-200, 100-250, 100-300, 100-350, 100-400, 100-500, 150-250, 150-300, 150-350, 150-400, 150-450, 150-500, etc.

The invention is generally disclosed herein using affirmative language to describe the numerous embodiments of the invention. The invention also specifically includes embodiments in which particular subject matter is excluded, in full or in part, such as substances or materials, method steps and conditions, protocols, or procedures. For example, in certain embodiments of the invention, materials and/or method steps are excluded. Thus, even though the invention is generally not expressed herein in terms of what the invention does not include aspects that are not expressly excluded in the invention are nevertheless disclosed herein.

A number of embodiments of the invention have been described. Nevertheless, one skilled in the art, without departing from the spirit and scope of the invention, can make various changes and modifications of the invention to adapt it to various usages and conditions. Accordingly, the following examples are intended to illustrate but not limit the scope of the invention claimed in any way.

### EXAMPLES

### Example 1

Adult Macaca mulatta non-human primates (males and females) were used in this study. Treatment groups were: Control (Vehicle Only); Low Dose (1.0×10¹³ vg/animal); Mid Dose (5.0×10¹³ vg/animal); and High Dose (2.17×10¹⁴ vg/animal).

Per Treatment Group Animal Numbers: Control (n=3 per timepoint), Low Dose (N=3 per timepoint), Mid Dose (n=3 per timepoint) and High Dose (n=4 per timepoint). Timepoints were 30 days and 90 days.

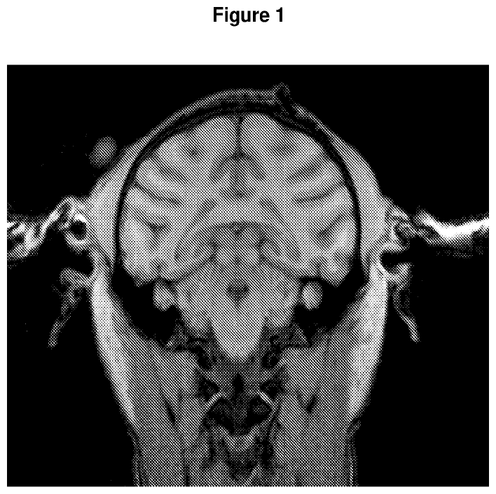

AAV2-CAG-hTTP1 Administration: MRI-guided unilateral delivery into the occipital horn of the lateral ventricle (FIG. 1; vertical line) using a spinal needle (22 G, 3.5″ Quinke BD). A total volume of 4 mL was delivered at (100 μL/min).

Cerebrospinal fluid (CSF) analysis included TPP1 enzymatic activity assay and human TPP1 protein expression assay (WES Western).

### Example 2

This example includes a description of data indicating short and long-term expression and activity of human TPP1 in CNS following intraventricular delivery of AAV2-CAG-humanTPP1.

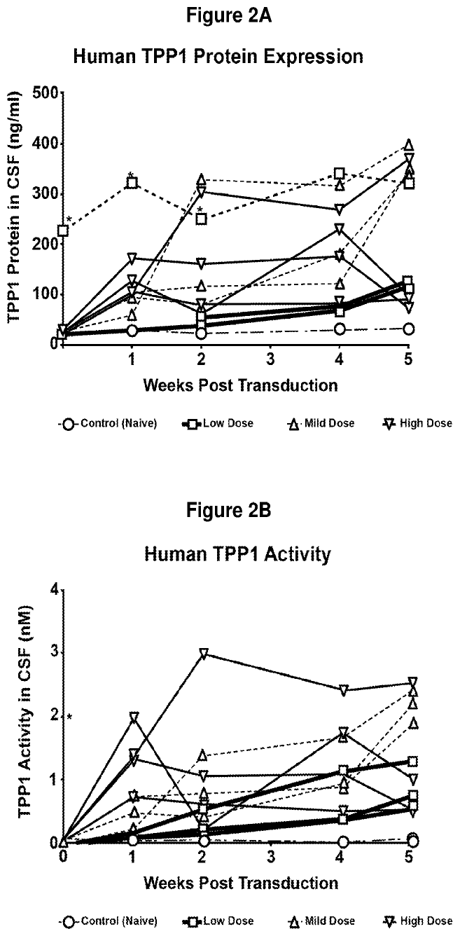

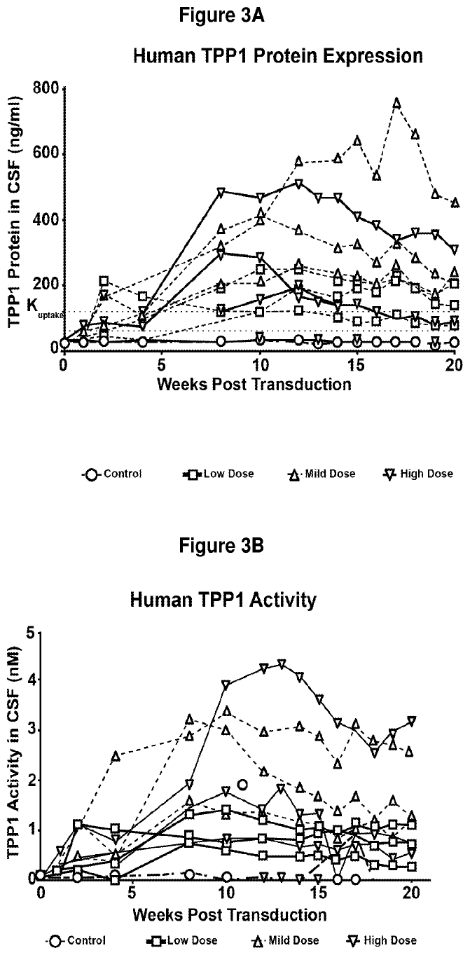

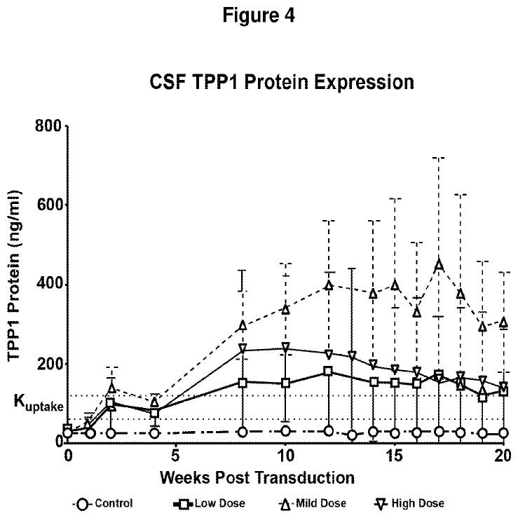

Human TPP was secreted into the CSF of non-human primates following delivery of an AAV-CAG-humanTPP1 (also referred to as AAV-CAG-hTPP1) vector targeting ependymal cells of the lateral ventricles in the CNS. Following delivery of the AAV vector, there was measurable and sustained expression of human TPP1 over a 20 week time-course (FIGS. 2A, 3A and 4). Moreover, TPP1 expression levels in all 3 AAV vector doses resulted in levels within or exceeding the Kᵤₚₜₐₖₑ for TPP1, as previously reported (Vuillemenot, B. R., et al. (2014) Toxicol Appl Pharmacol, 277(1), 49-57). This indicates that prolonged and continuous cellular uptake into the parenchyma in these animals is likely (Katz, M. L., et al. (2015) Sci Transl Med, 7(313); Tecedor, L. (2018) 16th International Conference on NCL, London, UK.). TPP1 activity analysis confirms the functional viability of the expressed TPP1 protein (FIGS. 2B and 3B). Preliminary analysis of post-mortem tissues in animals receiving AAV2-CAG-hTPP1, indicated no significant pathological changes compared to control animals receiving diluent only. Analysis of tissue uptake of expressed hTPP1 in the animals is being undertaken.

These studies demonstrate effective ependymal directed gene therapy approach that resulted in human TPP1 expression from the ependymal cells of the lateral ventricle for the treatment of late infantile neuronal ceroid lipofuscinosis.

### Example 3

Spk1 VP1 capsid (SEQ ID NO: 1):
MAADGYLPDWLEDNLSEGIREWWDLKPGAPKPKANQQKQDNGRGLVLPGYKYLGPFNGLDKG
EPVNAADAAALEHDKAYDQQLQAGDNPYLRYNHADAEFQERLQEDTSFGGNLGRAVFQAKKR
VLEPLGLVESPVKTAPGKKRPVEPSPQRSPDSSTGIGKKGQQPAKKRLNFGQTGDSESVPDP
QPIGEPPAAPSGVGPNTMAAGGGAPMADNNEGADGVGSSSGNWHCDSTWLGDRVITTSTRTW
ALPTYNNHLYKQISNGTSGGSTNDNTYFGYSTPWGYFDFNRFHCHFSPRDWQRLINNNWGFR
PKRLNFKLFNIQVKEVTQNEGTKTIANNLTSTIQVFTDSEYQLPYVLGSAHQGCLPPFPADV
FMIPQYGYLTLNNGSQAVGRSSFYCLEYFPSQMLRTGNNFEFSYNFEDVPFHSSYAHSQSLD
RLMNPLIDQYLYYLSRTQSTGGTAGTQQLLFSQAGPNNMSAQAKNWLPGPCYRQQRVSTTLS
QNNNSNFAWTGATKYHLNGRDSLVNPGVAMATHKDDEERFFPSSGVLMFGKQGAGKDNVDYS
SVMLTSEEEIKTTNPVATEQYGVVADNLQQQNAAPIVGAVNSQGALPGMVWQNRDVYLQGPI
WAKIPHTDGNFHPSPLMGGFGLKHPPPQILIKNTPVPADPPTTFNQAKLASFITQYSTGQVS
VEIEWELQKENSKRWNPEIQYTSNYYKSTNVDFAVNTEGTYSEPRPIGTRYLTRNL
Spk2 VP1 capsid (SEQ ID NO: 2):
MAADGYLPDWLEDNLSEGIREWWALQPGAPKPKANQQHQDNARGLVLPGYKYLGPGNGLDKG
EPVNAADAAALEHDKAYDQQLKAGDNPYLKYNHADAEFQERLKEDTSFGGNLGRAVFQAKKR
LLEPLGLVEEAAKTAPGKKRPVDQSPQEPDSSSGVGKSGKQPARKRLNFGQTGDSESVPDPQ
PLGEPPAAPTSLGSNTMASGGGAPMADNNEGADGVGNSSGNWHCDSQWLGDRVITTSTRTWA
LPTYNNHLYKQISSQSGASNDNHYFGYSTPWGYFDFNRFHCHFSPRDWQRLINNNWGFRPKK
LSFKLFNIQVKEVTQNDGTTTIANNLTSTVQVFTDSEYQLPYVLGSAHQGCLPPFPADVFMV
PQYGYLTLNNGSQAVGRSSFYCLEYFPSQMLRTGNNFQFSYTFEDVPFHSSYAHSQSLDRLM
NPLIDQYLYYLNRTQGTTSGTTNQSRLLFSQAGPQSMSLQARNWLPGPCYRQQRLSKTANDN
NNSNFPWTAASKYHLNGRDSLVNPGPAMASHKDDEEKFFPMHGNLIFGKEGTTASNAELDNV
MITDEEEIRTTNPVATEQYGTVANNLQSSNTAPTTRTVNDQGALPGMVWQDRDVYLQGPIWA
KIPHTDGHFHPSPLMGGFGLKHPPPQIMIKNTPVPANPPTTFSPAKFASFITQYSTGQVSVE
IEWELQKENSKRWNPEIQYTSNYNKSVNVDFTVDTNGVYSEPRPIGTRYLTRPL
CAG Promoter Sequence (SEQ ID NO: 3):
ATAGCCCATATATGGAGTTCCGCGTTACATAACTTACGGTAAATGGCCCGCCTGGCTGACCG
CCCAACGACCCCCGCCCATTGACGTCAATAATGACGTATGTTCCCATAGTAACGCCAATAGG
GACTTTCCATTGACGTCAATGGGTGGAGTATTTACGGTAAACTGCCCACTTGGCAGTACATC
AAGTGTATCATATGCCAAGTACGCCCCCTATTGACGTCAATGACGGTAAATGGCCCGCCTGG
CATTATGCCCAGTACATGACCTTATGGGACTTTCCTACTTGGCAGTACATCTACGTATTAGT
CATCGCTATTACCATGGTCGAGGTGAGCCCCACGTTCTGCTTCACTCTCCCCATCTCCCCCC
CCTCCCCACCCCCAATTTTGTATTTATTTATTTTTTAATTATTTTGTGCAGCGATGGGGGCG
GGGGGGGGGGGGGGGCGCGCGCCAGGCGGGGCGGGGCGGGGCGAGGGGCGGGGCGGGGCGAG
GCGGAGAGGTGCGGCGGCAGCCAATCAGAGCGGCGCGCTCCGAAAGTTTCCTTTTATGGCGA
GGCGGCGGCGGCGGCGGCCCTATAAAAAGCGAAGCGCGCGGCGGGCGGGGAGTCGCTGCGAC
GCTGCCTTCGCCCCGTGCCCCGCTCCGCCGCCGCCTCGCGCCGCCCGCCCCGGCTCTGACTG
ACCGCGTTACTCCCACAGGTGAGCGGGCGGGACGGCCCTTCTCCTCCGGGCTGTAATTAGCG
CTTGGTTTAATGACGGCTTGTTTCTTTTCTGTGGCTGCGTGAAAGCCTTGAGGGGCTCCGGG
AGGGCCCTTTGTGCGGGGGGAGCGGCTCGGGGGGTGCGTGCGTGTGTGTGTGCGTGGGGAGC
GCCGCGTGCGGCTCCGCGCTGCCCGGCGGCTGTGAGCGCTGCGGGCGCGGCGCGGGGCTTTG
TGCGCTCCGCAGTGTGCGCGAGGGGAGCGCGGCCGGGGGCGGTGCCCCGCGGTGCGGGGGGG
GCTGCGAGGGGAACAAAGGCTGCGTGCGGGGTGTGTGCGTGGGGGGGTGAGCAGGGGGTGTG
GGCGCGTCGGTCGGGCTGCAACCCCCCCTGCACCCCCCTCCCCGAGTTGCTGAGCACGGCCC
GGCTTCGGGTGCGGGGCTCCGTACGGGGCGTGGCGCGGGGCTCGCCGTGCCGGGCGGGGGGT
GGCGGCAGGTGGGGGTGCCGGGCGGGGCGGGGCCGCCTCGGGCCGGGGAGGGCTCGGGGGAG
GGGCGCGGCGGCCCCCGGAGCGCCGGCGGCTGTCGAGGCGCGGCGAGCCGCAGCCATTGCCT
TTTATGGTAATCGTGCGAGAGGGCGCAGGGACTTCCTTTGTCCCAAATCTGTGCGGAGCCGA
AATCTGGGAGGCGCCGCCGCACCCCCTCTAGCGGGCGCGGGGCGAAGCGGTGCGGCGCCGGC
AGGAAGGAAATGGGCGGGGAGGGCCTTCGTGCGTCGCCGCGCCGCCGTCCCCTTCTCCCTCT
CCAGCCTCGGGGCTGTCCGCGGGGGGACGGCTGCCTTCGGGGGGGACGGGGCAGGGCGGGGT
TCGGCTTCTGGCGTGTGACCGGCGGCTCTAGAGCCTCTGCTAACCATGTTCATGCCTTCTTC
TTTTTCCTACAGCTCCTGGGCAACGTGCTGGTTATTGTGCTGTCTCATCATTTTGGCAAA
TPP1 (SEQ ID NO: 4, Human):
MGLQACLLGLFALILSGKCSYSPEPDQRRTLPPGWVSLGRADPEEELSLTFALRQQNVERLS
ELVQAVSDPSSPQYGKYLTLENVADLVRPSPLTLHTVQKWLLAAGAQKCHSVITQDFLTCWL
SIRQAELLLPGAEFHHYVGGPTETHVVRSPHPYQLPQALAPHVDFVGGLHRFPPTSSLRQRP
EPQVTGTVGLHLGVTPSVIRKRYNLTSQDVGSGTSNNSQACAQFLEQYFHDSDLAQFMRLFG
GNFAHQASVARVVGQQGRGRAGIEASLDVQYLMSAGANISTWVYSSPGRHEGQEPFLQWLML
LSNESALPHVHTVSYGDDEDSLSSAYIQRVNTELMKAAARGLTLLFASGDSGAGCWSVSGRH
QFRPTFPASSPYVTTVGGTSFQEPFLITNEIVDYISGGGFSNVFPRPSYQEEAVTKFLSSSP
HLPPSSYFNASGRAYPDVAALSDGYWVVSNRVPIPWVSGTSASTPVFGGILSLINEHRILSG
RPPLGFLNPRLYQQHGAGLFDVTRGCHESCLDEEVEGQGFCSGPGWDPVTGWGTPNFPALLK
TLLNP

What is claimed is:

1. A method of treating a primate in need of tripeptidyl peptidase 1 (TPP1), comprising:
(a) providing a recombinant adeno-associated virus (AAV) vector comprising a nucleic acid encoding TPP1; and
(b) administering an amount of said recombinant AAV vector to the central nervous system (CNS) of said primate, wherein said TPP1 is expressed in said primate.

2. The method of claim 1, wherein said primate is a human.

3. The method of claim 2, wherein said human has late infantile neuronal ceroid lipofuscinosis (CLN2).

4. The method of claim 2, wherein said human is approximately 1-10 years old or is older than 10 years.

5. The method of claim 2, wherein said human is approximately 2-5 years old.

6. The method of any of claims 1-5, wherein said administration is to lateral ventricle or cisternae magna.

7. The method of claim 6, wherein said administration is to occipital horn of said lateral ventricle.

8. The method of any of claims 1-7, wherein said recombinant AAV vector is unilaterally administered to one lateral ventricle.

9. The method of any of claims 1-7, wherein said recombinant AAV vector is bilaterally administered to each lateral ventricle.

10. The method of any of claims 1-7, wherein said recombinant AAV vector is unilaterally or bilaterally administered to one or both lateral ventricles multiple times.

11. The method of any of claims 1-10, wherein said TPP1 is expressed at increased levels in said CNS.

12. The method of any of claims 1-11, wherein said TPP1 is expressed or delivered throughout the CNS.

13. The method of any of claims 1-12, wherein said TPP1 is expressed in or delivered to ependymal cells.

14. The method of any of claims 1-13, wherein said TPP1 is delivered to parenchyma.

15. The method of any of claims 1-14, wherein said TPP1 expression is sustained at levels equal to or greater than required for half maximal TPP1 uptake into neurons.

16. The method of any of claims 1-14, wherein said TPP1 expression is sustained at levels equal to or greater than Kᵤₚₜₐₖₑ, wherein Kᵤₚₜₐₖₑ is at least about 60 ng/mL.

17. The method of any of claims 1-14, wherein said TPP1 expression is sustained at levels equal to or greater than Kᵤₚₜₐₖₑ, wherein Kᵤₚₜₐₖₑ is at least about 60 ng/mL-120 ng/mL.

18. The method of any of claims 1-14, wherein said TPP1 expression is sustained at levels greater than about 120 ng/mL.

19. The method of any of claims 1-14, wherein said TPP1 expression is sustained at levels greater than about 150 ng/mL, greater than about 200 ng/mL, greater than about 250 ng/mL or greater than about 300 ng/mL.

20. The method of any of claims 1-19, wherein TPP1 expression is sustained for at least about 5 weeks, or at least about 10 weeks, or at least about 20 weeks in the CNS.

21. The method of any of claims 1-19, wherein detectable TPP1 expression or TPP1 activity is sustained for at least 5 weeks, or at least 10 weeks, or at least 20 weeks in the CNS.

22. The method of any of claims 1-21, wherein said recombinant AAV vector is administered to said CNS at a dose of greater than about 1.5×10¹³ AAV vector genomes; at a dose of about 5×10¹³ AAV vector genomes or greater than about 5×10¹³ AAV vector genomes; at a dose of about 1×10¹⁴ AAV vector genomes or greater than about 1×10¹⁴ AAV vector genomes; at a dose of about 5×10¹⁴ AAV vector genomes or greater than about 5×10¹⁴ AAV vector genomes; at a dose of about 1×10¹⁵ AAV vector genomes or greater than about 1×10¹⁵ AAV vector genomes; or at a dose of about 5×10¹⁵ AAV vector genomes or greater than about 5×10¹⁵ AAV vector genomes.

23. The method of any of claims 1-22, wherein said recombinant AAV vector is administered to said CNS at a dose range from about 1.5×10¹³ to about 5×10¹⁵ vector genomes; at a dose range from about 1×10¹⁴ to about 3×10¹⁵ vector genomes; at a dose range from about 2×10¹⁴ to about 2×10¹⁵ vector genomes; at a dose range from about 2.5×10¹⁴ to about 7.5×10¹⁴ vector genomes; at a dose range from about 5×10¹⁴ to about 5×10¹⁵ vector genomes; or at a dose range from about 1×10¹⁵ to about 5×10¹⁵ vector genomes.

24. The method of any of claims 1-22, wherein said recombinant AAV vector is administered to said CNS at a dose of about 1×10¹⁴ vector genomes, at a dose of about 2×10¹⁴ vector genomes, at a dose of about 3×10¹⁴ vector genomes, at a dose of about 4×10¹⁴ vector genomes, at a dose of about 5×10¹⁴ vector genomes, at a dose of about 6×10¹⁴ vector genomes, at a dose of about 7×10¹⁴ vector genomes, at a dose of about 8×10¹⁴ vector genomes, at a dose of about 9×10¹⁴ vector genomes, at a dose of about 1×10¹⁵ vector genomes, at a dose of about 2×10¹⁵ vector genomes, at a dose of about 3×10¹⁵ vector genomes, at a dose of about 4×10¹⁵ vector genomes, or at a dose of about 5×10¹⁵ vector genomes.

25. The method of any of claims 3-24, wherein said method reduces, decreases or inhibits one or more symptoms of said CLN2; or prevents or reduces progression or worsening of one or more symptoms of said CLN2; or stabilizes one or more symptoms of said CLN2; or improves one or more symptoms of said CLN2.

26. The method of claim 25, wherein said one or more symptoms is selected from the group consisting of: vision impairment, impaired or stunted cognitive development, loss of motor control and seizures.

27. The method of any of claims 1-26, wherein said nucleic acid encoding TPP1 comprises an expression cassette operably linked to an expression control element.

28. The method of claim 27, wherein said expression control element is positioned 5′ of said nucleic acid.

29. The method of claim 27 or 28, wherein said expression control element comprises a CAG (SEQ ID NO:3) promoter, cytomegalovirus (CMV) immediate early promoter/enhancer, Rous sarcoma virus (RSV) promoter/enhancer, SV40 promoter, dihydrofolate reductase (DHFR) promoter, or chicken β-actin (CBA) promoter.

30. The method of any of claims 1-29, wherein said heterologous nucleic acid is positioned between one or more 5′ and/or 3′ AAV inverted terminal repeats (ITR(s)).

31. The method of claim 30, wherein said one or more AAV ITR(s) comprises a mutated, modified or variant AAV ITR that is not processed by AAV Rep protein.

32. The method of claim 30, wherein said one or more AAV ITR(s) comprises a mutated, modified or variant AAV ITR that allows or facilitates formation of the self-complementary reporter transgene genome into a double strand inverted repeat sequence structure in said recombinant AAV vector.

33. The method of claim 32, wherein said mutated, modified or variant AAV ITR has a deleted D sequence, and/or a mutated, modified or variant terminal resolution site (TRS) sequence.

34. The method of any of claims 30-33, wherein said recombinant AAV vector comprises in 5′→3′ orientation a first AAV ITR; a promoter operable in mammalian cells; the heterologous nucleic acid; a polyadenylation signal; and optionally a second AAV ITR.

35. The method of any of claims 30-33, wherein said one or more ITR(s) comprises AAV serotype AAV1, AAV2, AAV3, AAV3B, AAV4, AAV5, AAV6, AAV7, AAV8, AAV9, AAV10, AAV11, AAV12, Rh74 or Rh10 ITR.

36. The method of any of claims 1-35, wherein said recombinant AAV vector comprises a VP1, VP2 or VP3 sequence 60% or more identical to a VP1, VP2 and/or VP3 sequence of AAV serotype AAV1, AAV2, AAV3, AAV3B, AAV4, AAV5, AAV6, AAV7, AAV8, AAV9, AAV10, AAV11, AAV12, Rh74, Rh10, SPK1 (SEQ ID NO:1), or SPK2 (SEQ ID NO:2) VP1, VP2 and/or VP3, or a hybrid or chimera of any of the foregoing AAV serotypes.

37. The method of any of claims 1-36, wherein said recombinant AAV vector comprises VP1, VP2 and/or VP3 capsid protein having 100% sequence identity to VP1, VP2 and/or VP3 capsid protein selected from the group consisting of AAV1, AAV2, AAV3, AAV3B, AAV4, AAV5, AAV6, AAV7, AAV8, AAV9, AAV10, Rh10, Rh74, SPK1 (SEQ ID NO:1) and SPK2 (SEQ ID NO:2) VP1, VP2 and/or VP3 capsid proteins.

38. The method of any of claims 1-37, wherein said recombinant AAV vector further comprises a polyadenylation sequence positioned 3′ of said nucleic acid.

39. The method of any of claims 1-38, wherein said nucleic acid encoding TPP1, expression control element or polyadenylation sequence is CpG reduced compared to wild-type nucleic acid encoding TPP1, expression control element or polyadenylation sequence.

40. The method of claim 38 or 39, wherein said polyadenylation sequence comprises a bovine growth hormone (bGH) polyadenylation sequence.

41. The method of any of claims 1-34, wherein said TPP1 is human, comprises or consists of the sequence set forth as SEQ ID NO:4, or is a functional variant or polymorphic form thereof.

42. The method of any of claims 1-41, wherein said recombinant AAV vector comprises:
(a) one or more of an AAV capsid, and
(b) one or more AAV inverted terminal repeats (ITR(s)), wherein said one or more AAV ITR(s) flanks the 5′ or 3′ terminus of said nucleic acid or said expression cassette.

43. The method of claim 42, further comprising an intron positioned 5′ or 3′ of said one or more ITR(s).

44. The method of claim 42 or 43, wherein at least one or more of said one or more ITR(s) and/or said intron is modified to have reduced CpGs.

45. The method of any of claims 1-44, wherein said recombinant AAV vector has a capsid serotype comprising an AAV VP1, VP2 and/or VP3 capsid having 90% or more sequence identity to AAV1, AAV2, AAV3, AAV3B, AAV4, AAV5, AAV6, AAV7, AAV8, AAV9, AAV10, AAV11, Rh10, Rh74, AAV-2i8, SPK1 (SEQ ID NO:1), or SPK2 (SEQ ID NO:2) VP1, VP2 and/or VP3 sequences, or a capsid having 95% or more sequence identity to AAV1, AAV2, AAV3, AAV3B, AAV4, AAV5, AAV6, AAV7, AAV8, AAV9, AAV10, AAV11, AAV12, Rh10, Rh74, AAV-2i8, SPK1 (SEQ ID NO:1), SPK2 (SEQ ID NO:2) VP1, VP2 and/or VP3 sequences, or a capsid having 100% sequence identity to AAV1, AAV2, AAV3, AAV3B, AAV4, AAV5, AAV6, AAV7, AAV8, AAV9, AAV10, AAV11, Rh10, Rh74, AAV-2i8, SPK1 (SEQ ID NO:1), or SPK2 (SEQ ID NO:2) VP1, VP2 and/or VP3 sequences.

46. The method of any of claims 41-46, wherein said one or more ITR(s) comprises one or more ITRs of any of: AAV1, AAV2, AAV3, AAV3B, AAV4, AAV5, AAV6, AAV7, AAV8, AAV9, AAV10, AAV11, Rh10, or Rh74 AAV serotypes, or a combination thereof.

47. The method of any of claims 1-46, wherein said recombinant AAV vector is in a pharmaceutical composition comprising a biologically compatible carrier or excipient.

48. The method of claim 47, wherein said pharmaceutical composition further comprises empty AAV capsids.

49. The method of claim 48, wherein the ratio of said empty AAV capsids to said recombinant AAV vector is within or between about 100:1-50:1, from about 50:1-25:1, from about 25:1-10:1, from about 10:1-1:1, from about 1:1-1:10, from about 1:10-1:25, from about 1:25-1:50, or from about 1:50-1:100.

50. The method of claim 48, wherein the ratio of said empty AAV capsids to said recombinant AAV vector is about 2:1, 3:1, 4:1, 5:1, 6:1, 7:1, 8:1, 9:1, or 10:1.

51. The method of any of claims 47-50, wherein said pharmaceutical composition further comprises a surfactant.
